(19) United States
(12) Patent Application Publication
(10) Pub. No.: US 2009/0138038 A1
(43) Pub. Date: May 28, 2009

(54) EMBOLIC FILTER DEVICE AND METHOD OF MANUFACTURING THE SAME

(75) Inventor: Patrick Griffin, Castlegar (IE)

Correspondence Address: Sughrue Mion-ABBOTT LABS, 2100 Pennsylvania Avenue, N.W., Washington, DC, 20037, US

(73) Assignee: SALVIAC LIMITED, Dublin (IE)

(21) Appl. No.: 12/256,556

(22) Filed: Oct. 23, 2008

Related U.S. Application Data
(60) Provisional application No. 60/983,171, filed on Oct. 27, 2007.

(51) Int. Cl. A61F 2/06

(52) U.S. Cl. 606/200

(57) ABSTRACT

An embolic filter device and method of manufacturing the same are provided. The method includes reducing a wall thickness of a tube in a central region between a proximal end portion and a distal end portion of the tube; cutting the proximal end portion of the tube to form an annular ring at a proximal end of the proximal end portion; cutting a plurality of slits in the central region; and expanding a portion of the tube between the annular ring and the distal end portion. The device includes a distal annular ring; a mesh connected to the distal annular ring; and a proximal annular ring connected to the mesh by at least two connecting portions.

### BACKGROUND OF THE INVENTION

1. Field of the Invention

Devices and methods consistent with the present invention relate to filters for filtering plaque and other occlusions in blood vessels and, more particularly, to an embolic filter device formed as a single component for filtering plaque and other occlusions dislodged during treatment of stenosis in a vasculature.

2. Description of the Related Art

Arteriosclerosis, also known as atherosclerosis, is a common human ailment arising from the deposition of fatty-like substances, referred to as atheroma or plaque, on the walls of blood vessels. Such deposits occur in peripheral blood vessels that feed limbs of the body, coronary blood vessels that feed the heart, and in carotid blood vessels that feed the head, neck, and brain. Localized accumulation of deposits within regions of the blood vessels may result in stenosis, or narrowing of the vascular channel. When this occurs, blood flow is restricted and the person's health is at serious risk.

Numerous approaches for reducing and removing such vascular deposits have been proposed, including balloon angioplasty, in which a balloon-tipped catheter is used to dilate a stenosed region within the blood vessel; atherectomy, in which a blade or other cutting element is used to sever and remove the stenotic material; laser angioplasty, in which laser energy is used to ablate at least a portion of the stenotic material; and the like.

During treatment using the above-described approaches, a filter element is deployed downstream of a treatment area, e.g., in the case of a treatment area in the carotid artery, between the treatment area and the brain, in order to filter and remove any pieces of plaque or occlusion material which may be dislodged during treatment and thus enter the brain causing a stroke or other damage.

There are a number of different designs of related art filter elements. One example of a related art filter element is an umbrella type filter comprising a membrane supported on a collapsible frame on a guidewire for movement of the filter membrane between a collapsed position against the guidewire and a laterally extending position occluding a blood vessel. Examples of such filters are shown in U.S. Pat. No. 4,723,549, U.S. Pat. No. 5,053,008, U.S. Pat. No. 5,108,419, and WO 199833443, each of the disclosures of which are incorporated by reference herein in its entirety.

Another example of a related art filter element is disclosed in U.S. Pat. No. 6,336,934, the disclosure of which is herein incorporated by reference in its entirety. The related art filter element disclosed on U.S. Pat. No. 6,336,934 comprises a compressible porous structure polymeric foam filter element overmoulded onto or joined to a polymeric or metallic tube or spring or other hollow support element.

Related art filter elements, such as, for example, those described above, suffer from a number of problems. First, the related art filter devices have a two component structure, i.e., a support frame and a filter media mounted outside the support frame. This configuration increases the delivery profile of the device. Second, the two components must be joined using, for example, an adhesive joint. This process increases the number of steps and complexity of the manufacturing process required to produce the overall device assembly. Third, the filter media of the related art filter element is not radio-opaque, making the filter element more difficult to locate when the filter element is inside the vasculature of a patient, thus increasing the complexity of the treatment procedure and increasing the potential for causing treatment errors.

### SUMMARY OF THE INVENTION

Exemplary embodiments of the present invention address the above disadvantages and other disadvantages not described above. Also, the present invention is not required to overcome the disadvantages described above.

An object of the present invention is to provide an embolic filter device having a reduced delivery profile.

Another object of the present invention is to provide an embolic filter device having a simplified manufacturing process.

Yet another object of the invention is to provide an embolic filter device which is easier to locate inside the vasculature of a patient.

According to an aspect of the present invention, there is provided a method of manufacturing an embolic filter device, the method including reducing a wall thickness of a tube in a central region between a proximal end portion and a distal end portion of the tube; cutting the proximal end portion of the tube to form an annular ring at a proximal end of the proximal end portion; cutting a plurality of slits in the central region; and expanding a portion of the tube between the annular ring and the distal end portion.

According to another aspect of the present invention, there is provided a filter device for filtering blood in blood vessels, the filter device including a distal annular ring; a mesh connected to the distal annular ring; and a proximal annular ring connected to the mesh by at least two connecting portions.

According to yet another aspect of the present invention, there is provided a filter device for filtering blood in blood vessels, the filter device having an expanded state and an unexpanded state, the device including, in the unexpanded state, an annular ring; a central region which has a plurality of slits cut therein and is connected to the annual ring by at least two connecting portions; and a distal end portion which is connected to the central region, and wherein in the expanded state, the central region expands to form a mesh.

### BRIEF DESCRIPTION OF THE DRAWINGS

The above and other aspects of the present invention will become more apparent by describing in detail exemplary embodiments thereof with reference to the attached drawings, in which:

FIGS. 1A-1E are views showing a method of manufacturing an embolic protection filter according to an exemplary embodiment of the present invention;

FIGS. 2A and 2B are sectional views of details A and B, respectively, of FIG. 1C;

FIGS. 3A and 3B are close-up views of detail C of FIGS. 1D and 1E, respectively, according to an exemplary embodiment of the present invention;

FIGS. 4A and 4B are close-up views of detail D of FIGS. 1D and 1E, respectively, according to an exemplary embodiment of the present invention.

### DESCRIPTION OF EXEMPLARY EMBODIMENTS OF THE PRESENT INVENTION

Hereinafter, exemplary embodiments of the present inventive concept will be described in detail with reference to the drawings. The same reference numbers are used to denote the same elements even in different drawings.

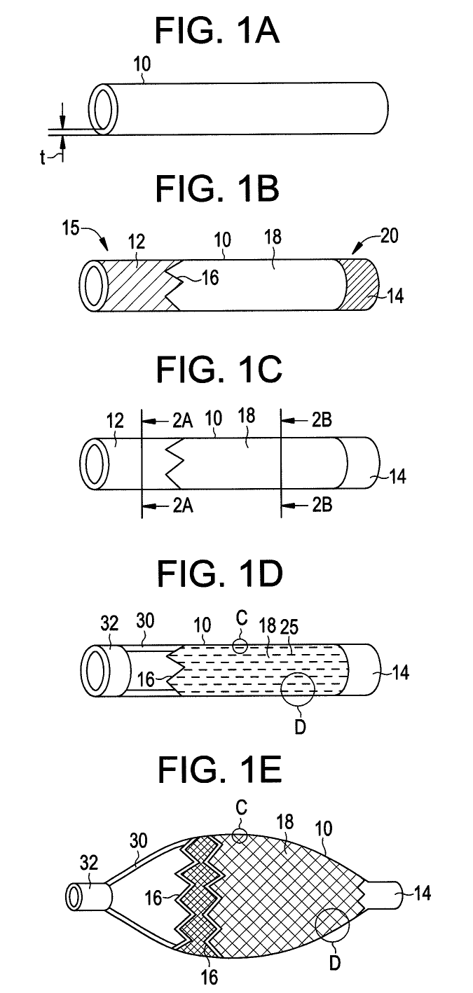

Referring to FIGS. 1A to 1E, a method of manufacturing an embolic protection filter according to an exemplary embodiment of the present invention is shown. The method begins in FIG. 1A with a tube 10. The tube 10 is hollow and has a wall thickness t. The tube is made of nitinol or other material having physical properties similar to nitinol.

As shown in FIG. 1B, tube 10 has a proximal end 15 and a distal end 20. A proximal end portion 12 of the tube 10 and a distal end portion 14 of the tube 10 are masked to create a central region 18 which remains unmasked. The proximal end portion 12 may be masked in a pattern 16, as shown in FIG. 1B.

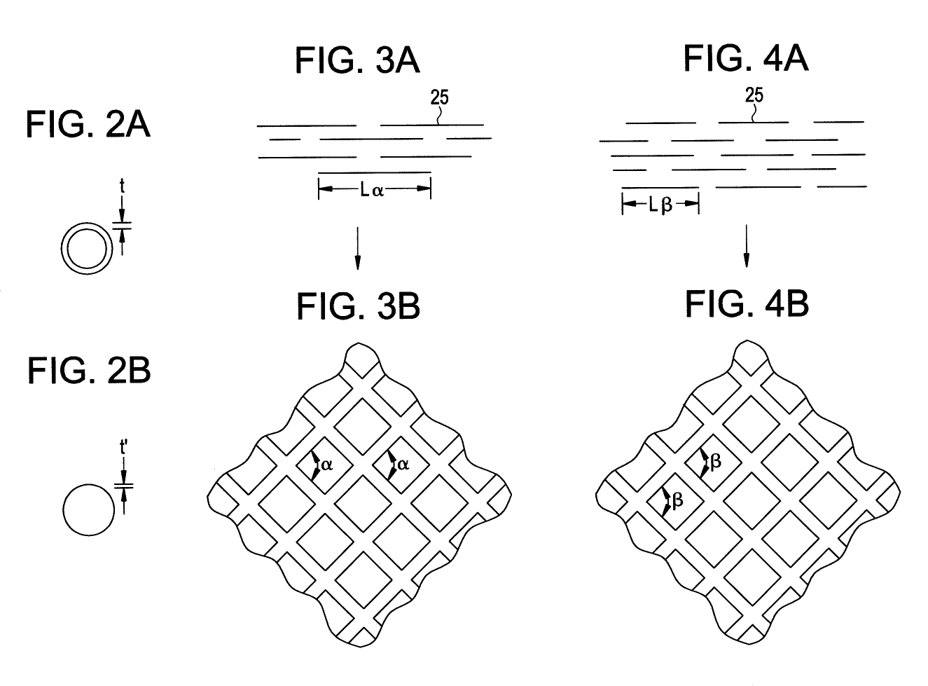

The central region 18 is then etched to reduce the wall thickness in the central region 18. FIG. 2A shows a cross-section along line A-A of the proximal end section 12 of FIG. 1C. FIG. 2B shows a cross-section along line B-B of the central region 18 of FIG. 1C. As may be seen from comparing FIGS. 2A and 2B, the wall thickness t′ in the central region 18, as shown in FIG. 2B, is less than the wall thickness t of the masked portions of the tube 10, as shown in FIG. 2A. In other words, t′<t. In this exemplary embodiment, the etching is performed by photoetching. However, other known etching processes may also be used.

Turning to FIG. 1D, a plurality of slits 25 are cut in the central region 18. The slits 25 are cut parallel to an axis of the tube 10 and are spaced in a uniform pitch radially around the body of the tube 10 in the central region 18. The proximal end portion 12 is cut to form an annular ring 32 at a proximal end of the proximal end portion 12, the annular ring 32 being attached to the central region 18 by at least two connecting portions 30. The distal end portion 14 also forms an annular ring at the distal end of the tube 10.

The central region 18 is then expanded to form the filter, as shown in FIG. 1E. During the process of expansion, the slits 25 are each opened, thus forming a mesh throughout the central region 18 of the tube 10. The annular ring 32 at the proximal end 15 of the tube 10, and the distal end portion 14 at the distal end 20 of the tube 10 are not expanded, and thus maintain their original form. The annular ring 32 at the proximal end 15 of the tube 10 is connected to the expanded central region 18 by the at least two connecting portions 30.

As discussed above, the slits 25 are expanded to form a mesh, and the mesh serves to filter plaque and other occlusion material as the blood flows through the mesh. A length of the slits 25 determines a grain of the mesh, once the slits 25 are expanded, i.e., how fine the mesh is in a given area of the central region 18.

Turning to FIGS. 3A and 3B, the slits 25 of detail C of FIGS. 1D and 1E, respectively, i.e., at a proximal side of the central region 18, are shown. FIG. 3A shows the slits 25 in an unexpanded configuration, while FIG. 3B shows the slits 25 that have been expanded to form a mesh. A length Lα of the slits 25 in FIG. 3A results in an angle α in each open region of the mesh, as shown in FIG. 3B.

Turning to FIGS. 4A and 4B, the slits 25 at detail D of FIGS. 1D and 1E, i.e., at a distal side of the central region 18, are shown. FIG. 4A shows the slits 25 in an unexpanded configuration, and FIG. 4B shows the slits 15 that have been expanded into the mesh. A length Lβ of the slits 25 in the distal side of the central region 18 results in an angle β in each open region of the mesh.

The grain of the mesh may be controlled by adjusting the lengths of the slits 25. A longer length of the slits 25 results in a larger angle in the open parts of the mesh, and therefore a more coarse grained mesh, whereas a shorter length of the slits 25 results in a smaller angle and therefore a more fine grained mesh. In this exemplary embodiment, a length Lα of the slits 25 at a proximal side of the central region 18 is longer than a length Lβ of the slits 25 at a distal side of the central region 18. However, Lα may also be set equal to Lβ, in which case the slits 25 would be of a uniform length throughout the central region 18. The length of the slits 25 may also be varied to gradually become shorter from the proximal end to the distal end of the central region 18. This would result in a mesh which becomes gradually more fine as the distal end of the central region 18 is approached.

Due to the physical properties of the nitinol used to manufacture the tube 10, once the tube 10 is expanded to form the mesh which serves the filtering function, the filter may be compressed and inserted into a delivery catheter. The delivery catheter is then inserted into the femoral artery of a patient and advanced to a downstream side of a treatment area. The filter is then delivery from the delivery catheter into the blood vessel of a patient, where the filter expands to its originally expanded shape. In this way, the filter expands to the size of the blood vessel to be filtered, and the filter performs its filtering function.

The foregoing exemplary embodiments and advantages are merely exemplary and are not to be construed as limiting the present invention. The present teaching can be readily applied to other types of apparatuses. Also, the description of the exemplary embodiments of the present invention is intended to be illustrative, and not to limit the scope of the claims, and many alternatives, modifications, and variations will be apparent to those skilled in the art.

What is claimed is:

1. A method of manufacturing an embolic filter device, the method comprising:
reducing a wall thickness of a tube in a region between a proximal end portion and a distal end portion of the tube to form a thin-walled region;
cutting the proximal end portion of the tube to form an annular ring at a proximal end of the proximal end portion;
cutting a plurality of slits in the region between the proximal end portion and the distal end portion; and
expanding a portion of the tube between the annular ring and the distal end portion.

2. The method of claim 1, wherein reducing the wall thickness of the tube comprises:
masking the proximal end portion and the distal end portion of the tube; and
etching a central portion of the tube to reduce a wall thickness thereof.

3. The method of claim 2, wherein the masking is performed using a photo etch mask.

4. The method of claim 4, wherein the etching is performed by photo etching.

5. The method of claim 1, wherein the cutting is performed using a laser.

6. The method of claim 1, wherein the tube is a metal tube made of nitinol.

7. The method of claim 1, wherein the plurality of slits are cut in a uniform pitch radially around a body of the tube.

8. The method of claim 7, wherein a length of the plurality of slits becomes progressively shorter from the proximal end portion to the distal end portion.

9. The method of claim 1, wherein a length of each of the plurality of slits in a proximal region of the thin-walled region is Lα, and a length of each of the plurality of slits in a distal region of the thin-walled region is Lβ, and Lα is greater than Lβ.

10. The method of claim 1, wherein a length of each of the plurality of slits is constant.

11. A filter device for filtering blood in blood vessels, the filter device comprising:
a distal annular ring;
a mesh connected to the distal annular ring; and
a proximal annular ring connected to the mesh by at least two connecting portions.

12. The filter device of claim 11, wherein the distal annular ring, the proximal annular ring, the mesh, and the at least two connecting portions are nitinol.

13. The filter device of claim 11, wherein a grain of the mesh becomes progressively more fine from a proximal end to a distal end of the mesh.

14. The filter device of claim 11, wherein a grain of the mesh is uniform.

15. A filter device for filtering blood in blood vessels, the filter device having an expanded state and an unexpanded state, the device comprising:
in the unexpanded state:
an annular ring;
a region which has a plurality of slits cut therein and is connected to the annual ring by at least two connecting portions; and
a distal end portion which is connected to the region, and wherein in the expanded state, the region expands to form a mesh.

16. The filter device of claim 15, wherein the annular ring, the region, the mesh, and the at least two connecting portions are nitinol.

17. The filter device of claim 15, wherein a grain of the mesh becomes progressively more fine from a proximal end to a distal end of the mesh.

18. The filter device of claim 15, wherein a grain of the mesh is uniform.
